SEQUENCE LISTING (25 sequences)

SEQ ID NO: 1 - DNA, 19 bp, Homo sapiens
gttggagctggtgacgtag

SEQ ID NO: 2 - DNA, 19 bp, Homo sapiens
gcgcgctttgtaggattcg

SEQ ID NO: 3 - DNA, 19 bp, Homo sapiens
gagacctacctccggatca

SEQ ID NO: 4 - DNA, 21 bp, Homo sapiens
gtgcgctgctggtgccaaccc

SEQ ID NO: 5 - DNA, 20 bp, Artificial
  Primer for U6-KrasD13 siRNA (forward)
gaactagtggatccgacgcc

SEQ ID NO: 6 - DNA, 81 bp, Artificial
  Primer for U6-KrasD13 siRNA (reverse)
ggatccaaaaaagttggagctggtgacgtagtctcttgaactacgtcaccagctccaacaaacaaggcttttctccaaggg

SEQ ID NO: 7 - DNA, 26 bp, Artificial
  Primer for U6-luc siRNA (forward)
ggatccaaggtcgggcaggaagaggg

SEQ ID NO: 8 - DNA, 82 bp, Artificial
  Primer for U6-luc siRNA (reverse)
ggatccaaaaaagtgcgctgctggtgccaaccctctcttgaagggttggcaccagcagcgcacggtgtttcgtcctttccac

SEQ ID NO: 9 - DNA, 34 bp, Artificial
  Primer for amplification of human U6 promoter
tgctggatccaagcttaaggtcgggcaggaagag

SEQ ID NO: 10 - DNA, 40 bp, Artificial
  Primer for amplification of human U6 promoter
gcatgctcgaggccggccgatatataaagccaagaaatcg

SEQ ID NO: 11 - DNA, 26 bp, Artificial
  Primer for amplification of murine U6 promoter
tctagagaactagtggatccgacgcc

SEQ ID NO: 12 - DNA, 40 bp, Artificial
  Primer for amplification of murine U6 promoter
gccgctcgaggcgcgccatatttatagtctcaaaacacac

SEQ ID NO: 13 - DNA, 15 bp, Artificial
  5′ flanking sequence for siRNA vector
ccaggacgacaaaaa

SEQ ID NO: 14 - DNA, 16 bp, Artificial
  3′ flanking sequence for siRNA vector
atttttggcttttcgg

SEQ ID NO: 15 - DNA, 20 bp, Artificial
  Adaptor oligo hU6/FseI
ctttttgtcgtcctggccgg

SEQ ID NO: 16 - DNA, 21 bp, Artificial
  Adaptor oligo mU6/AscI
cgcgccgaaaagccaaaaata

SEQ ID NO: 17 - DNA, 19 bp, Artificial
  Sense strand siRNA against mTOR
gagaggaaaggtggcatct

SEQ ID NO: 18 - DNA, 21 bp, Artificial
  Sense strand siRNA against mTOR
gagccacatcagaccttatat

SEQ ID NO: 19 - DNA, 21 bp, Artificial
  Sense strand siRNA against mTOR
gcctattctgaaggcattaat

SEQ ID NO: 20 - DNA, 22 bp, Artificial
  Sense strand siRNA against Hsp90
gcgcatctatcgcatgatcaat

SEQ ID NO: 21 - DNA, 20 bp, Artificial
  Sense strand siRNA against Hsp90
gcggtaaggataagaagaat

SEQ ID NO: 22 - DNA, 21 bp, Artificial
  Sense strand siRNA against Hsp90
gttggacagtggtaaagagct

SEQ ID NO: 23 - DNA, 20 bp, Artificial
  Sense strand siRNA against PLK
gagacctacctccggatcat

SEQ ID NO: 24 - DNA, 21 bp, Artificial
  Sense strand siRNA against PLK
gacagcctgcagtacatagat

SEQ ID NO: 25 - DNA, 21 bp, Artificial
  Sense strand siRNA against PLK
ggtgtatcatgtataccttgt

(19) United States
(12) Patent Application Publication
(10) Pub. No.: US 2006/0234249 A1
(43) Pub. Date: Oct. 19, 2006

(54) Novel assays for assessing cancerous cell growth

(75) Inventors: Ning Ke, San Diego, CA (US); De-Hua Yu, San Diego, CA (US); Aaron Albers, Berkeley, CA (US); Gisela Claassen, San Diego, CA (US); Henry (Qi-Xiang) Li, Carlsbad, CA (US); Flossie Wong-Staal, San Diego, CA (US)

Correspondence Address: LAW OFFICE OF DAVID SPOLTER, 1590 COAST WALK, LA JOLLA, CA, 92037, US

(73) Assignee: Immusol Incorporated, San Diego, CA (US)

(21) Appl. No.: 11/106,990

(22) Filed: Apr. 15, 2005

(51) Int. Cl. C12Q 1/68

(52) U.S. Cl. 435/6

(57) ABSTRACT

The present invention provides novel assays for assessing cancerous cell growth. The invention is useful for the identification and validation of oncogenes and tumor suppressors, as well as for the identification and validation of therapeutic compounds for the treatment of cancer.

### FIELD OF THE INVENTION

The present invention provides novel assays for assessing cancerous cell growth. The invention is useful for the identification and validation of oncogenes and tumor suppressors, as well as for the identification and validation of therapeutic compounds for the treatment of cancer.

### BACKGROUND INFORMATION

Anchorage-independent growth, the cell's ability to proliferate without attachment to, or spreading onto, a substratum, is one of the hallmarks of the transformation process characteristic of cancer. It is the most accurate in vitro indication of tumorigenicity (Shin et al., Proc. Natl. Acad. Sci. USA, 72:4435-4439 (1975); Fiebig et al., Europe J. of Cancer, 40:802-820 (2004)) and the most commonly used criterion for the validation of genes involved in cancer.

The primary method of monitoring anchorage-independent growth is manual counting of colonies growing in semisolid culture media (soft agar). However, the traditional soft agar assay is time-consuming, labor-intensive, and plagued with inconsistencies due to subjectivity in the counting process.

More specifically, this traditional assay is laborious both in the initial setup and in the final quantification stages. For even a limited number of samples, the experiment is quite cumbersome, demanding a great deal of incubator space due to the traditional method's rather bulky scale. Also, the duration of the assay is long. For example, three or four weeks or growth are usually required to observe any phenotypic change. Furthermore, the necessarily long culture period significantly increases the chances of contamination. Lastly, the traditional counting of colonies by eye is subject to bias. Moreover, colony size is difficult, if not impossible, to determine meaningfully.

In an attempt to improve on these limitations, it has been suggested to use high throughput methods based upon liquid culture poly (2-hydroxyethylmethacryl-ate) (poly-HEMA; Sigma, St. Louis, Mo., USA)-coated microplates. Fukazawa et al., Anal. Biochem., 228:83-90 (1995). However, this method has certain limitations. Firstly, it is difficult to coat the plates homogeneously, which is important to ensure consistent results. Secondly, the variant growth assay is not widely accepted as a substitute for measuring growth in semisolid medium, perhaps explaining why there are no such commercially coated plates available. Finally, evidence suggests that this method may not measure the exact same oncological properties as the traditional soft agar assay.

Another drawback of current assay methodologies is that they are not suited to the high throughput screening (HTS) techniques used today both for target validation and compound screening. While current genomics tools (including global gene expression profiling) can identify a large number of candidates as potential drug targets, phenotypic validation of these candidates is a long and laborious process. Usually, at the end of this process, very few of the candidates are found to actually possess disease-modifying properties.

Accordingly, there is now a bottleneck in identifying and validating drug targets. Thus, a more efficient and reliable assay for identifying and validating drug targets and therapeutic compounds is needed.

Furthermore, traditional methodologies are also inefficient when used in connection with gene silencing by means of siRNA, which has become a critical tool in modern target validation technologies. Stable expression of siRNAs, for example, via retroviral vector, is plagued by the very nature of oncogene targets: their inactivation causes cell death or reduced growth, hindering generation of stable cell lines. In addition, generating stable cell lines can be lengthy and labor-intensive, and cannot readily be adapted to HTS multiplexed for a large number of targets and siRNAs.

The present invention addresses these limitations, and provides additional advantages as well.

### SUMMARY OF THE INVENTION

The present invention provides novel assays for assessing cancerous cell growth.

In one aspect, the invention provides a cell survival assay adapted for identifying and/or validating a gene involved in cancer, the assay including: (i) a substrate having a multiplicity of wells, each well having a semi-solid medium therein; (ii) cancer cells seeded in the semi-solid medium; (iii) an agent that interacts with the cancer cells; (iv) a detectable marker; and (v) means for automatically quantifying cell survival resulting from the interaction between the agent and the cancer cells.

In another aspect, the invention provides a cell survival assay for identifying and/or validating a therapeutic agent for use in the treatment of cancer, the assay including: (i) a substrate having a multiplicity of wells, each well having a semi-solid medium therein; (ii) cancer cells seeded in the semi-solid medium; (iii) an agent that interacts with the cancer cells; (iv) a detectable marker; and (v) means for automatically quantifying cell survival resulting from the interaction between the agent and the cancer cells.

In yet another aspect, the invention provides a high throughput screening method for identifying and/or validating a gene involved in cancer growth, the method including: (a) seeding cancer cells in a semi-solid medium, within the wells of a substrate having a multiplicity of wells; (b) introducing into the wells a detectable marker and an agent that interacts with the cancer cells; (c) allowing the cells to grow for between about 5 and about 15 days; and (d) obtaining automatically a quantifiable measure of cell survival or cell growth resulting from the interaction between the agent and the cancer cells.

In yet another aspect, the invention provides a high throughput screening method for identifying and/or validating a compound as a therapeutic agent useful for the treatment of cancer, the method including: (a) seeding cancer cells in a semi-solid medium, within the wells of a substrate having a multiplicity of wells; (b) introducing into the wells a detectable marker and a compound that interacts with the cancer cells; (c) allowing the cells to grow for between about 5 to about 15 days; and (d) obtaining automatically a quantifiable measure of cell survival or cell growth resulting from the interaction between the compound and the cancer cells.

### BRIEF DESCRIPTION OF THE DRAWINGS

FIG. 1 is a graph showing the linear correlation between number of live cells in culture media (both liquid culture and soft agar)and a readout by means of alamarBlue™ staining. HeLa or HeLaHF cells at various densities were seeded into wells of a 96-well microplate in either liquid media (FIG. 1A) or soft agar (FIG. 1B) Growth was scored by alamarBlue™ staining with excitation at 530 nm and emission at 590 nm (in liquid culture after one day and in soft agar culture after seven days). The x-axis represents the number of cells plated, and the y-axis represents the alamarBlue™ reading corrected for background. Error bars indicate standard deviations. When not shown, the standard deviation is within the size of the symbols.

FIG. 2A is a bar graph showing soft agar growth in wells of a 96-well plate in accordance with the invention of cells transiently transfected with short interfering RNA (siRNA) targeted against two different oncogenes (Kras^{D13} and PLK). DLD-1 or HeLa cells were transiently transfected with 10 nM of in vitro transcribed siRNAs against Kras^{D13} (DLD-1) or polo-like kinase (PLK; HeLa) and control siRNA using Oligofectamine™ reagent. After transfection (24 h), the cells were trypsinized, and 1000 cells/well were seeded into soft agar media and allowed to grow for seven days before being scored by alamarBlue™ staining. Each sample was performed in triplicate; error bars indicate standard deviations. P=0.01 for Kras^{D13} in DLD-1 cells, and P<0.01 for PLK in HeLa cells.

FIG. 2B is a bar graph showing relative growth of DLD-1 cells stably transduced with either short interfering RNA (siRNA) against KrasD13 or a control siRNA, and grown either in a conventional 10-cm dish or in wells of a 96-well plate in accordance with the assay of the present invention. For the 10-cm dish, 5000 cells were plated in soft agar and allowed to grow for 3 weeks before being scored by Qcount™; for the 96-well plate, 1000 cells per well of each type were seeded into soft agar and allowed to grow for 1 week before being scored by alamarBlue™ staining. Each sample was performed in triplicate. Error bars indicate standard deviations, P=0.03 for the 10-cm plate and P<0.01 for the 96-well assays.

FIG. 3 shows the linear correlation between luciferase activity and the number of live cells in liquid culture. PC3/luc cells (PC3 cells stably transduced with the gene expressing luciferase) were seeded into wells of a 96-well plate in liquid culture at varying densities. Cell growth in culture was scored after incubation at 37° C. for one day both by measuring luciferase activity (FIG. 3A) and by alamarBlue™ staining (excitation at 530±25 nm and emission at 590±35 nm; FIG. 3B). The x-axis represents the number of cells seeded, and the y-axis represents either the relative luciferase activity in units per second (RLU) (FIG. 3A) or the alamarBlue™ reading (FIG. 3B). Each sample was in triplicate; error bars indicate standard deviations.

FIG. 4 shows the linear correlation between luciferase activity and the number of live cells in soft agar culture. PC3/luc cells were seeded into wells of a 96-well plate in soft agar culture at varying densities. After incubation at 37° C. for 4 hrs, both luciferase activity (FIG. 4A) and alamarBlue™ staining (FIG. 4B) were measured. Each sample was in triplicate; error bars indicate standard deviations.

FIG. 5 shows the structure of the opposing promoter siRNA gene expression cassette (pHUMU) which may be used in the practice of the invention; also shown is the sequence of an siRNA (mTor2319) targeting the oncogene mTor which may be expressed using this expression cassette.

FIG. 6A is a bar graph showing relative cell survival/proliferation reduction of Hela cells after transfection with three different siRNAs targeting three different oncogenes (Hsp90β, mTor and PLK), or with cDNA expressing a tumor suppressor (Bax), and co-transfection with either luciferase or β-galactosidase. Also shown is cell survival/proliferation reduction based upon alamarBlue™ staining.

FIG. 6B is a bar growth showing results of the same experiment as shown in FIG. 6A, except that A549 cells were used, and co-transfection only with luciferase.

FIG. 7 is a bar growth showing results of the screening of a test compound (IMST 10) in PC3 cells using the assay of the present invention.

### DETAILED DESCRIPTION

The present invention provides novel assays for measuring cancer cell growth. The assays of the invention are useful for identifying and validating a gene involved in cancer (for example, an oncogene or a tumor suppressor), or for screening for a compound which may have therapeutic use in the treatment of cancer. In one aspect, an assay in accordance with the invention comprises (i) a substrate having a multiplicity of wells, each well having a semi-solid medium therein; (ii) cancer cells seeded in the semi-solid medium; (iii) an agent that interacts with the cancer cells; (iv)a detectable marker; and (v) means for automatically quantifying the results of the interaction between the agent and the cancer cells growing in the wells.

In the practice of the invention, the interaction between the agent and the cancer cells may occur on the nucleic acid level, and typically will involve inhibiting the expression of a gene of interest expressed in the cells. Accordingly, the agent which may be used within the scope of the invention may be a nucleic acid molecule such as an siRNA, a ribozyme or an antisense molecule, any of which may target, bind to, or inactivate the mRNA of the gene of interest expressed in the cells. Such nucleic acid molecules may be introduced into the cells by any means known in the art, including transfection or viral vector transduction.

The interaction between the agent and the cancer cells may also occur on the protein level. Accordingly, the agent which may be used within the scope of the invention may be an antibody, or an antibody fragment, or a peptide.

The assay in accordance with the invention may also be used for the screening of therapeutic compounds potentially useful in the treatment of cancer. Thus, the agent which interacts with the cancer cells in accordance with the invention may be a small molecule, an antibody, an antibody fragment, a peptide, or any other compound that may affect the growth of the cancer cells and could be used as a therapeutic agent.

Any detectable marker known in the art may be used within the scope of the assay comprising the present invention. The detectable marker may be, for example, a compound which can be detected using fluorometric or calorimetric means, such as alamarBlue™, tertazolium salt or WST-1. The detectable marker may be introduced into the cells by any means, including transfection or transduction through a vector, for example, the transfection or viral vector transduction of a reporter gene, such as luciferase or β-galactosidase (lacZ).

The means for automatically quantifying the results of the interaction between the agent and the cancer cells growing in the semi-solid medium in the wells, in accordance with the invention, may be any suitable device known in the art, including various forms of “plate readers” which provide a readout of the amount and/or activity of the detectable compound present in the live cells in each of the wells. Examples of such devices include the CytoFluor® Series 400 Multi-Well Plate Reader manufactured by PerSeptive Biosystems, Framingham, Mass.; and the Mithras LB 940 luminometer manufactured by Berthold Technology, Bad Wildbad, Germany.

The substrate having a multiplicity of wells in accordance with the invention preferably is a microplate, which typically has 96 wells. However, any multi-well plate may be used within the scope of the invention, for examples, plates with 6, 12, 24, 48, 384 or more wells.

The present invention further provides a high throughput screening method for identifying and/or validating a gene involved in cancer growth. The method comprises: (a) seeding cancer cells in a semi-solid medium, within the wells of a substrate having a multiplicity of wells; (b) introducing into the wells a detectable compound and an agent that interacts with the cancer cells; (c) allowing the cancer cells to grow for between about 5 to about 15 days; and (d) obtaining automatically a quantifiable measure of the results of the interaction between the agent and the cancer cells growing in the wells. Generally, the cells can be grown for about 5 to about 20 days. Preferably, the cells are grown for at least about 7 days. More preferably, they are grown between about 7 and about 15 days. Even more preferably, they are grown about 7 to about 12 days.

In yet another aspect, the invention provides a high throughput screening method for identifying and/or validating a compound as a therapeutic agent useful for the treatment of cancer, the method including: (a) seeding cancer cells in a semi-solid medium, within the wells of a substrate having a multiplicity of wells; (b) introducing into the wells a detectable marker and a compound that interacts with the cancer cells; (c) allowing the cells to grow for between about 5 to about 15 days; and (d) obtaining automatically a quantifiable measure of cell survival or cell growth resulting from the interaction between the compound and the cancer cells. Generally, the cells can be grown for about 5 to about 20 days. Preferably, the cells are grown for at least about 7 days. More preferably, they are grown between about 7 and about 15 days. Even more preferably, they are grown about 7 to about 12 days.

The methods of the present invention typically can involve silencing or inhibiting a gene of interest expressed in the cells. Accordingly, the agent which may be used within the scope of the invention may be a nucleic acid molecule such as an siRNA, a ribozyme or an antisense molecule, any of which may target, bind to, or inactivate the mRNA of the gene of interest expressed in the cells. Such nucleic acid molecules may be introduced into the cells by any means known in the art, including transfection or viral vector transduction.

The agent which is introduced into the cancer cells in the practice of the present invention may interact with the cells on the protein level. Accordingly, the agent which may be used within the scope of the invention may be an antibody, or an antibody fragment, or a peptide.

As described above, the method of the present invention may also be used for screening therapeutic compounds potentially useful in the treatment of cancer. When used for this purpose, the agent which is introduced into the cancer cells and which interacts with them may be any small molecule compound, an antibody, an antibody fragment, a peptide, or any other compound that may affect the growth of the cancer cells and could be used as a therapeutic agent.

In the practice of the methods of the invention, any detectable compound known in the art may be used. Typically, the detectable compound will be a compound which can be detected using fluorometric or colorimetric means, such as alamarBlue™, tertazolium salt or WST-1. The detectable compound may be introduced into the cells by any means, including transfection or transduction through a vector, for example, the transfection or transduction of a reporter gene, such as luciferase or β-galactosidase.

In the practice of the methods of the invention, any device which automatically provides a readout of the amount and/or activity of the detectable compound present in each of the wells may be used. Examples of such devices include various forms of “plate readers” such as the CytoFluor® Series 400 Multi-Well Plate Reader manufactured by PerSeptive Biosystems, Framingham, Mass.; and the Mithras LB 940 luminometer manufactured by Berthold Technology, Bad Wildbad, Germany.

The method of the invention is particularly useful for assays based upon “transient transfection”, for example, transient transfection of siRNA molecules. As used herein, “transient transfection” means the process of introducing an agent into cells and measuring the growth or proliferation of the cells within a relatively brief interval of time. Typically, this interval is within about seven days, although the interval may be up to about 15 days or as little as about one day.

As used herein, “semi-solid medium” means a medium for growing cells that is between the solid and liquid state. To achieve such a medium, agar, for example, can be added in low concentrations to achieve a low level of fluidity (typically 0.3-0.6% w/v), but not added at such a concentration that will result in a firm gel. Liquid media allow cells to move around freely, and if the cells require attachment for survival (i.e. are “anchorage-dependent”), they will attach to the culture vessel. Semi-solid media, in contrast, are media with less fluidity such that they prevent cells from moving freely and therefore only support cell populations in suspension. Once the cells are in suspension, they cannot move around freely and will not be able to attach to the culture vessel. Certain cells, e.g. cancer cells, can grow in such media, and are therefore deemed “anchorage-independent”.

The term “semi-solid medium” includes the growth media that are necessary to keep the cells alive and provide the nutrients necessary for them to grow, such as Dulbecco's Modified Eagle's Medium (DMEM), fetal bovine serum, essential and non-essential amino acids, and the like, as is known in the art.

Motility Test Medium is one example of a semi-solid medium. Another example of a semi-solid medium is soft agar. Soft agar assays typically involve two or three layers of semi-solid matrix, with each layer formed by mixing agar or agarose with a liquid nutrient medium, at a suitable temperature. Each layer may be of about equal volume, although the bottom layer may have a slightly higher concentration of semi-solid matrix, such as agarose, and may contain everything contained in the top layer except the cells.

The two most commonly used soft agar assay techniques are referred to as the Hamburger-Salmon (or H-S) and the Courtenay-Mills (or C-M) methods. In the Hamburger-Salmon assay, enriched media are added to agar with the nutrients being added to both layers, with cells included in the top layer. The bottom layer consists of about 0.5% agar and the top layer is of about 0.3% agar. The cultures are plated in dishes (plates containing one to 24 wells per plate).

An alternative method is described by Courtenay-Mills, see for example, Courtenay, V. D., Recent Results in Cancer Research, 94:17-34 (1984), in which red blood cells are added to the agar as a component in the agar layers, requires the addition of liquid medium about every 5 days, at low atmosphere, and uses culture tubes, instead of plates.

Methylcellulose is also a semi-solid medium for suspension cell growth which may be used in the assay to facilitate harvesting of the colonies. Addition of methylcellulose is traditionally used when culturing erythroid progenitor cells. The viscosity of methylcellulose prevents aggregation of the cells, but is less effective in holding the cells in place. In contrast, agar and agarose, even at a percentage as low as 0.3%, form true gels.

Various semi-solid media have been examined. In one study of lymphoma cells, purified agarose was found to be superior than agar or methylcellulose for determining colony formation and inhibitory effects (Hays, et al. In Vitro Cellular & Developmental Biology, 21:266-270 (1985). Agarose, which is a modified agar with most of the large charged molecules removed, may allow for better diffusion of growth factors than agar with its charged matrix molecules.

To circumvent the problem of needing larger well volume for the soft agar assays, researchers have tested other semi-solid media to mimic cell growth in soft agar. As an example, in a study reported by Fukazawa, H., et al. 1995, “A microplate assay for quantitation of anchorage-independent growth of transformed cells”, Analytical Biochemistry 228:83-90, poly(HEMA)-coated 96-well plates were assessed by tetrazolium dye reduction or 3H-thymidine incorporation. Poly(HEMA) is an anti-adhesive polymer with results similar to soft agar (only looking at cell growth, no compound effects).

Another adaptation to the traditional soft agar assay involves quantitating colonies by use of 3H-thymidine incorporation into DNA as the cell divides, instead of relying on visual counting to determine the number of colonies. In this particular assay, red alga extract Furcellaran is used as the substrate gel in a single layer as opposed to the double layer semi-solid matrix system.

Typically, the preferred semi-solid media for use in the present invention form a liquid at temperatures above room temperature and above the temperature required to incubate the cells, and form a semi-solid, or gel, when at about room temperature or the temperature at which the cells are incubated. Although agar and agarose are preferred, a wide variety of polymers, including proteins and their derivatives, may be used as semi-solid media in the practice of the present invention. Matrigel®, collagen or gelatin, or other similar materials may also be used as the semi-solid media.

Agar is a generic name for a class of compounds generally defined as a dried mucilaginous substance extracted from red algae, having the property of melting at about 100 deg. C. and solidifying into a gel at about 40 deg. C. Agar is not digested by most bacteria and is used as a gel in the preparation of solid culture media. Dorland's Illustrated Medical Dictionary, 25th ed., W. B. Saunders Co. 1974.

Agarose is the neutral linear polysaccharide fraction found in agar preparations, generally comprised of D-galactose and altered 3,6-anhydrogalactose residues; thus agarose is a modified agar. Agarose is a purified linear glactan hydrocolloid isolated from agar or agar-bearing marine algae. Agarose forms a gel matrix when it is at its gel point, which may be different than its melting temperature. Typically, sugars, methyl groups, and other chemical groups are chemically bonded to agar, or fraction derived from agar, in order to enhance desired physical properties, such as low gelling temperature.

Collagen is a major mammalian protein of the white fibers of connective tissues, cartilage, and bone. Collagen is generally insoluble in water, but is typically altered to improve desirable properties such gelling at a given temperature.

Gelatin is a derived protein formed from collagen of tissues by boiling in water. Gelatin swells up when put in cold water, but dissolves in hot water.

In the case of Matrigel®, collagen or gelatin, or other similar materials, temperature control of the material is important to prevent premature gelling when the material warms to about room temperature. Therefore, with such materials, it is important to keep the materials chilled prior to filling the wells.

The wells of the multi-well substrate may contain a single layer of the semi-solid medium, or they may contain multiple layers. In one embodiment, each well contains multiple layers of the same semi-solid medium. In another embodiment, the wells may contain layers of different types of semi-solid media, for example, the bottom layer may comprise gelatin, and an upper layer may comprise a gelatin-agarose mixture. Where different types of semi-solid media are used in the wells, it is preferred that the bottom layers have a density greater than the density of the upper layers.

The present invention provides a highly efficient and reliable method to screen for putative oncogenes (or tumor suppressors) in a high throughput format. For example, as shown in Example 2 below, the assay of the present invention confirmed the role of K-RAS as an oncogene. K-RAS belongs to the RAS family of GTPases and is a classical dominant oncogene. Its activation, usually via point mutations around the GTP binding site, causes cell transformation (e.g., elevated anchorage-independent growth). siRNA has recently been shown to down-regulate gene expression effectively, including KrasV12 mutant allele in pancreatic cancer cells, CAPAN-1 cells (7,8,10-12). Brummelkamp, et al., Cancer Cell., 2:243-247 (2002); Lee, et al., Nat. Biotechnol., 20:500-505 (2002); Elbashir, et al., Nature, 411:494-498 (2001); Miyagishi, et al., Nat. Biotechnol., 20:497-500 (2002); and Paul, et al., Nat. Biotechnol., 20:505-508 (2002).

In light of this, a siRNA lentiviral vector against mutant K-RAS^{D13} was constructed and the vector delivered into DLD-1 cells by transduction. Cells stably expressing the siRNA constructs were then subjected to TaqMan® real-time reverse transcription PCR (RT-PCR) analysis and the targeted message was shown to have been down-regulated.

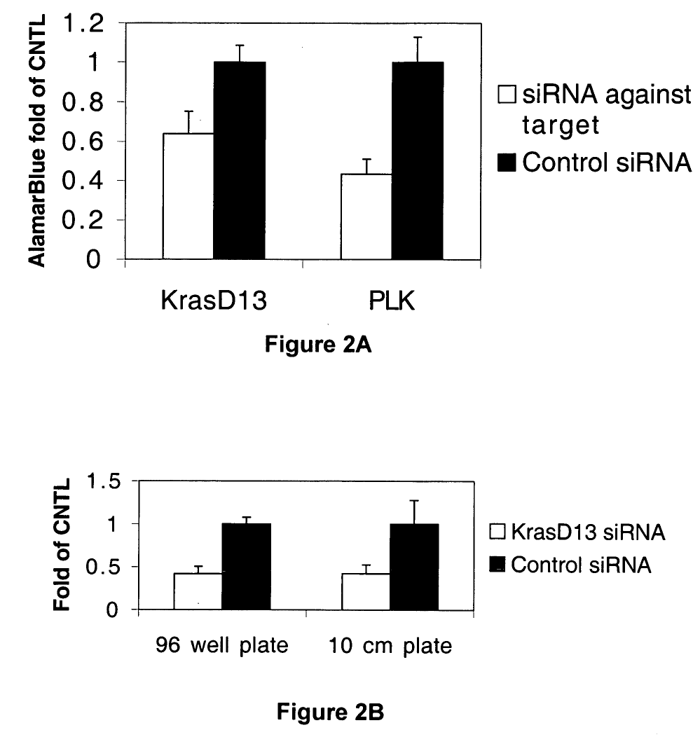

Soft agar growth assays following the stable transduction were set up in both traditional 10-cm dishes and 96-well plates. The cells in the 10-cm dishes were cultured for 3 weeks and the colonies were quantitated by Qcount® to avoid human counting subjectivity; the cells in the wells of the 96-well plate were cultured for one week and cell survival was measured by a plate reader based upon alamarBlue™ staining (alamarBlue™ contains an oxidation-reduction indicator that both fluoresces and changes color in response to chemical reduction of growth medium resulting from cell growth). The cells transduced with lentiviral vector expressing siRNA against mutant K-RAS were shown to be significantly lower in anchorage-independent growth in both 96-well plates (as measured by alamarBlue™ staining) and 10-cm dishes (as measured by number of colonies) (both by 60%; FIG. 2B), due to the down-regulation of this oncogene. These results demonstrate the correlation between the high throughput assay of the invention and the traditional soft agar growth assay in monitoring real oncogene transformation potential.

Moreover, the traditional soft agar growth assay is difficult to adapt for use in connection with transient transfection experiments, due to the assay's long incubation time. However, the assay of the present invention, with an incubation time of just one week or less, makes such adaptation possible, as shown in Example 2.

SiRNA targeting Kras^{D13} or PLK was transcribed in vitro, along with a nonspecific control siRNA, and delivered into DLD-1 cells by transient transfection. The transfected cells were plated into soft agar media 24 hours after transfection. Similar to the stable siRNA expression experiment described above (FIG. 2B), transient transfection of Kras^{D13} siRNA caused a 30% reduction in anchorage-independent growth and transient transfection of PLK siRNA caused a reduction of 50% in anchorage-independent growth (FIG. 2A). These results not only confirm the oncogenic properties of Kras^{D13} and PLK, but also demonstrate the usefulness of the assay of the present invention in oncology target validation.

The utility of the assay of the present invention in connection with transient transfection of siRNA is significant because transient transfection has very important implications in large-scale target screening due to the difficulty and expense of constructing siRNA expression vectors in large numbers by cloning. Thus, a convenient alternative to such construction is to generate siRNAs in parallel by chemical synthesis or in vitro transcription, which requires transient transfection for the cell-based assay.

The assay of the present invention may also be effectively adapted for use in connection with the screening of compounds having anti-cancer properties. A typical protocol for such an assay would provide as follows:

A basal feeder layer of 1.2% agar in IMDM cell culture medium is prepared and added at 50 μl/well in a 96-well tissue culture plate. 60 μl of the cell layer consisting of 1000 cells/well in 0.8% agar in IMDM medium is then overlayed after the feeder layer solidifies. Liquid culture plates to measure anchorage dependent growth are also set up in parallel using cell growth media keeping the volumes and cell numbers the same. Several serial fold dilutions of each compound to be tested are prepared in a separate plate including “no compound” controls and then added to the soft agar plate. Each compound is added in a 40 μl volume over the solidified cell layer such that the final volume in each well is 150 μl. Additional controls include DMSO or the vehicle used to dissolve the compound. Each dilution of each compound is plated in triplicate. Plates are incubated at 37 degrees C. for one week after which alamarBlue™ staining is performed to test for cell viability. Data are represented as mean fold of vehicle (DMSO) control from triplicate samples with standard deviation.

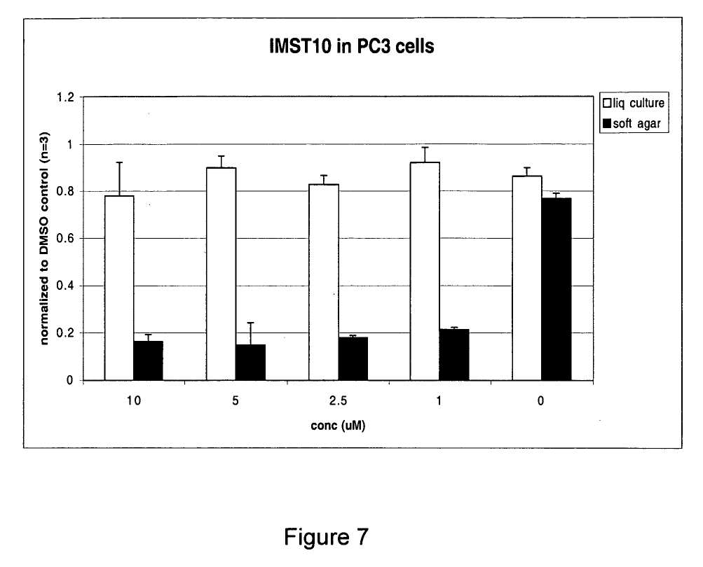

FIG. 7 is a bar graph showing the results of just such an assay performed with PC3 cells in connection with a compound code-named IMST10. The results show that various concentrations of this compound do not affect cancer cell growth in liquid culture, but do have a marked effect on anchorage-independent growth as measured in accordance with the assay of the invention in soft agar. These results indicate that this compound is not toxic to normal cells, but is effective in killing cancer cells, such that it may have use as a therapeutic compound for the treatment of cancer.

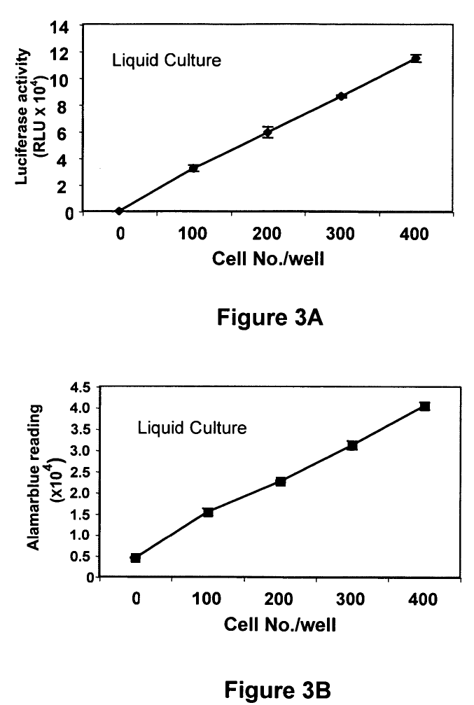

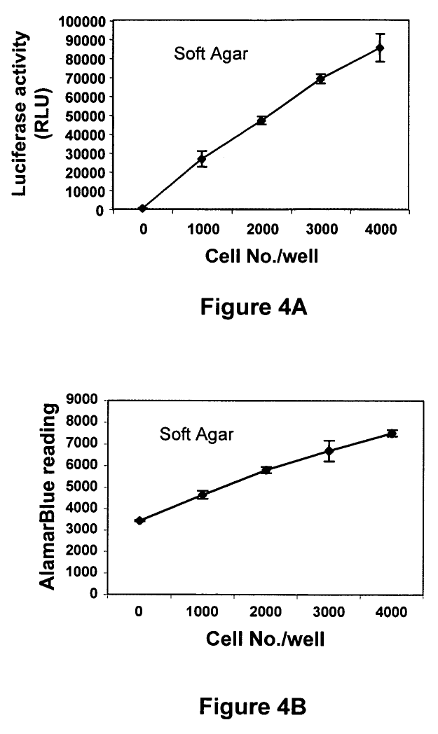

A further embodiment of the present invention is described in greater detail in Examples 3 and 4. These show that measurement of cancer cell survival and growth may be effectively done in a high throughput format by co-transfecting a viral vector expressing siRNA and a reporter gene expressing measurable markers such as luciferase or β-galactosidase. The co-transfection may be done using either two separate vectors, or a single vector expressing both the siRNA and the reporter gene. The readout of cell survival based upon the expression of the reporter gene correlates very well with the readout obtained using alamarBlue™ when all the surviving cells contain the reporter (see FIGS. 3 and 4). Moreover, since the readout of the reporter only reflects cells that have survived transfection with siRNA, it has a significantly higher signal-to-noise ratio, regardless of transfection efficiency. Thus, the results obtained using this methodology are much more sensitive than those obtained based upon alamarBlue™.

In summary, the present invention provides several advantages. First, it shortens incubation time, making transient transfection possible. Second, it provides automatic quantification and analysis, leading to reliable results and eliminating experimental bias. Third, it is easily adaptable to parallel operation, which increases validation speed and overall productivity. Fourth, it can be adapted for use with many different cancer cell lines. Finally, it can be effectively used not only for target discovery and validation, but also for the screening of therapeutic anti-cancer compounds.

The following examples are intended to illustrate but not limit the subject invention.

### EXAMPLES

### Example 1

This example shows that, using the assay of the present invention, anchorage-independent growth can be reliably and accurately measured in a high throughput system with substantially reduced incubation time.

A. Materials And Methods

Cells HeLa is a cervical carcinoma cell line, and HeLaHF is a HeLa revertant with a loss-of-transformation phenotype. Boylan, et al., Cell Growth Differ., 7:725-735 (1996). DLD-1 is a human colon carcinoma cell line.

HeLa cells are known to form colonies in soft agar media and form tumors in nude mice, while the non-transformed derivative, HeLaHF, does not. The growth of HeLa and its non-transformed derivative, HeLaHF, was therefore compared in both normal liquid media and semisolid soft agar media in 96-well microplates.

All cells were cultured in Dulbecco's modified Eagle's medium (DMEM), 10% fetal bovine serum (FBS), 2 mM L-Glutamine (L-Glu), nonessential amino acids (NEAAs), and 1% sodium pyruvate (all from Invitrogen, Carlsbad, Calif., USA) in a humidified incubator (Ultra-Tech WJ301D; Baxter Scientific Products, West Chester, Pa., USA) with 5% CO₂ at 37° C.

Anchorage-dependent Growth (Normal Liquid Cell Culture) in 96-well Plates

Cell suspensions (100 μL) containing 0-2×10³ cells were plated into wells of a 96-well flat-bottom microplate. The cells were allowed to grow for 1-3 days before the cell growth was measured using alamarBlue™ staining (1:10 volume reagent; Biosource International, Camarillo, Calif., USA), according to the manufacturer's instructions.

Anchorage-independent Growth (Soft Agar Cell Culture) in 96-well Plates

A mixture of 25 μL prewarmed (37° C.) 2×Iscove's modification of Dulbecco's medium (IMDM) containing 20% FBS, 4 mM L-Glu, 2×NEAA, 0.6% sodium bicarbonate, 2% sodium pyruvate, 200 U/mL penicillin/streptomycin (Invitrogen), and 25 μL prewarmed (56° C.) 1.2% Bacto™ Agar Select (BD Biosciences, San Jose, Calif., USA) were plated onto wells of a 96-well microplate to serve as a prelayer for the assay. Ten microliters of cell suspensions containing 0-2×10³ cells were mixed with 20 μL 2×IMDM and 30 μL 0.8% Bacto Agar Select in a 96-well round-bottom polypropylene microplate and transferred to the 96-well microplate containing the solidified prelayers. Semisolid feeder layers were then prepared by mixing 25 μL 2×IMDM and 25 μL 1.2% Bacto Agar Select and layered on top of the solidified cell layers.

The cells were allowed to grow in a humidified 37° C. incubator with 5% CO₂ for 1-2 weeks before cell proliferation and viability were scored using alamarBlue™. The read-out was obtained using a CytoFluor® Series 4000 Multi-Well Plate Reader (PerSeptive Biosystems, Framingham, Mass., USA), with excitation at 530 nm and emission at 590 nm.

Anchorage-independent Growth (Soft agar Cell Culture) in a 10-cm Plate

A mixture of 2 mL of prewarmed (37° C.) 2×IMDM and 3 mL prewarmed (56° C.) 0.8% Bacto Agar Select per plate (0.4% final agar) were mixed with 1 mL cell suspension and seeded over a 0.6% agar/IMDM prelayer (8 mL) in a 10-cm dish. Semisolid 0.6% feeder layers (6 mL) were overlayed on top of the solidified cell layers. The cells were allowed to grow in the humidified 37° C. incubator with 5% CO₂ for 21-28 days. Colony numbers were determined by Qcountm (Spiral Technology, Norwood, Mass., USA).

B. Results

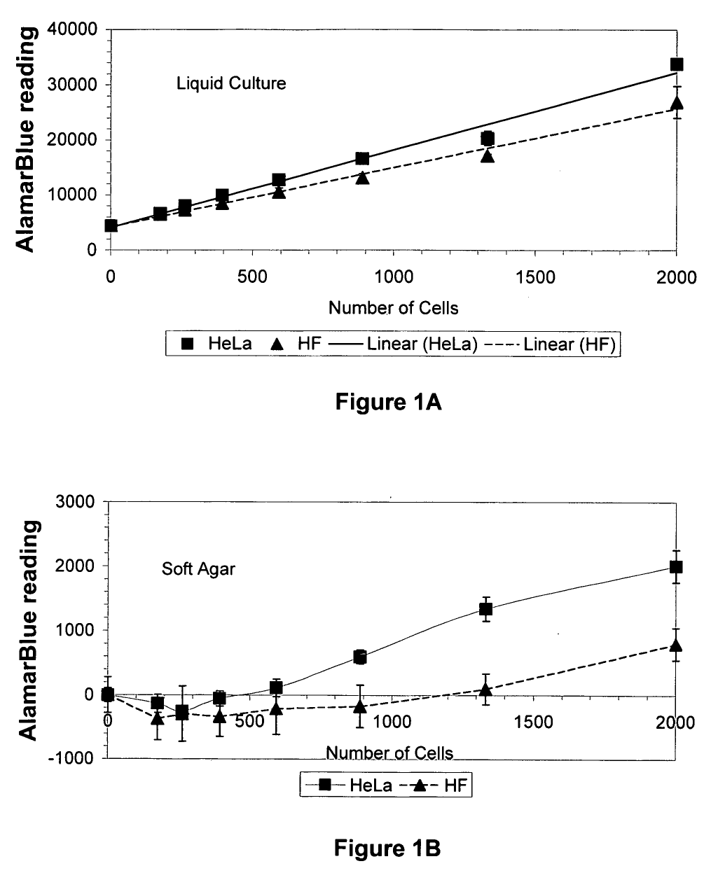

In liquid culture (anchorage-dependent growth), a linear relationship between alamarBlue™ readings and cell number was observed for both HeLa and HeLaHF in the range from 0-2×10³ cells (FIG. 1A). The revertant HeLaHF cells showed slightly slower growth in liquid culture compared to the transformed HeLa cells. A similar linear relationship for anchorage-independent growth was also observed for HeLa in the range from 6×10² to 2×10³ cells/well, while the reading for HeLaHF was not above the background for up to 1.4×10³ cells (FIG. 1B).

These results were consistent with the distinct anchorage-independent growth potential of each member of this isogenic cell pair, as observed in the traditional soft agar colony (10 cm plate; 21-28 day growth) assay. Thus, this example shows that anchorage-independent growth can be reliably and accurately measured in a high throughput system with substantially reduced incubation time. In addition, these results provide a wide working range of cell densities (for example, 6×10² to 1.4×10³ cells/well) that could be used to distinguish cells with different anchorage-independent growth potential.

Several other cancer cell lines that grow in soft agar media using this higher throughput format were also measured, including A431 (epidermoid cancer), A549 (lung cancer), DLD-1 (colon cancer), DU145 (prostate cancer), HCT116 (colon cancer), MCF7 (breast cancer), and U87 (glioma). Similar observations were obtained in connection with these cell lines, with a wide variation in optimal seeding density, which indicates that. the method of the present invention may be effectively used in connection with a large number of cell types.

### Example 2

This example shows that the assay of the present invention may be utilized for the validation of genes involved in cancer, and that it is effective both when the cells are transiently transfected by in vitro transcribed siRNA and when the cells are stably transduced with siRNA by means of a viral vector.

A. Materials and Methods

Cells

HeLa and DLD1 cells were prepared as described above in Example 1.

Transient Transfection of in vitro Transcribed siRNA

siRNA against KrasD3 , polo-like kinase (PLK), and a nonspecific sequence control siRNA were prepared using the Gene-Silencer™ siRNA Construction Kit (Ambion, Austin, Tex., USA), following the manufacturer's instructions. The target sequences are listed in Table 1.

HeLa or DLD1 cells (5×10³ or 1×10⁴, respectively) were seeded on day 1 and transfected on day 2 with 10 nM of siRNA using Oligofectamine™ transfection reagent (Invitrogen), according to the manufacturer's recommendations. On day 3 (24 h post-transfection), the cells were trypsinized and a desired number seeded into either liquid culture or soft agar culture (prepared as described above in Example 1) in wells of a 96-well plate.

Transduction of Cells with Lentiviral Vector Expressing siRNAs

The pHIV-7 vector (City of Hope, Duarte, Calif., USA) was constructed from pHIV-7-GFP by digestion with PstI to remove the cytomegalovirus (CMV) promoter/enhanced green fluorescent protein (EGFP) cDNA cassette and religation of the 530- and 5.7-kb fragments. Banerjea et al., Mol. Ther., 8:62-71 (2003); Yam et al., Mol. Ther., 5:479-484 (2002).

A 1.4-kb PvuII/BamHI fragment comprising the simian virus 40 (SV40) promoter/puro^{r} cassette was isolated from the pPUR vector (BD Biosciences Clontech, Palo Alto, Calif., USA), treated with Klenow to repair the BamHI-digested 3′ overhang, and ligated into the SmaI site of pHIV-7 to yield pHIV-7-puro^{r}. Forward orientation of the SV40 promoter/puro^{r} cassette was confirmed by digestion with BamHI and XbaI to yield a 1.2-kb fragment.

Target sequences for the Kras^{D13} (Brummelkamp et al., Cancer Cell., 2:243-247 (2002)) and control (luciferase) siRNAs are listed in Table 1.

Coding sequences for hairpin siRNAs were appended to U6 promoters by PCR amplification. The U6 promoter sequences in psilencer (Ambion) and pTZ U6+1 (Lee, et al., Nat. Biotechnol., 20:500-505 (2002)) were used as templates for the construction of expression cassettes for Kras^{D13} and control (luciferase) siRNAs, respectively.

PCR primers comprising the siRNA coding sequences are also shown in Table 1. PCR was performed using Platinum PCR SuperMix High Fidelity (Invitrogen). The PCR products were ligated into the pCR Blunt® II-TOPO® vector (Invitrogen), sequenced in both directions, digested with BamHI, and ligated into BamHI-digested pHIV-7-puro. The reverse orientation (i.e., U6 and SV40 promoters facing in opposite directions) was selected for both vectors.

Vesicular stomatitis virus G glycoprotein (VSV-G) pseudotyped lentiviral vector was packaged in 293FT cells using the ViraPower™ Lentiviral Packaging Mix (both from Invitrogen). DLD1 cells were transduced using standard methods and subjected to selection with puromycin for 7-10 days. Tiscornia, et al., Proc. Natl. Acad. Sci. USA, 100:1844-1848 (2003). A desired number of cells were seeded into either the traditional or the 96-well soft agar culture for the assay.

TABLE 1

| | |
|---|---|
| Name | Sequence |

Targets for In Vitro Transcribed siRNA

| | |
|---|---|
| KrasD13 | 5′-GTTGGAGCTGGTGACGTAG-3′ (SEQ ID NO:1) |
| Control | 5′-GCGCGCTTTGTAGGATTCG-3′ (SEQ ID NO:2) |
| PLK | 5′-GAGACCTACCTCCGGATCA-3′ (SEQ ID NO:3) |

Targets for Lentiviral-Expressed siRNA

| | |
|---|---|
| KrasD13 | 5′-GTTGGAGCTGGTGACGTAG-3′ (SEQ ID NO:1) |
| Control | 5′-GTGCGCTGCTGGTGCCAACCC-3′ (SEQ ID NO:4) |

Primers for Amplification of U6-siRNA Cassettes

| | |
|---|---|
| U6-KrasD13 | 5′-GAACTAGTGGATCCGACGCC-3′ (SEQ ID NO:5) |
| siRNA (forward) | |
| U6-KrasD13 | 5′-GGATCCAAAAAAGTTGGAGCTGGTGAC |
| siRNA (reverse) | GTAGTCTCTTGAACTACGTCACCAGCTCCAA |
| | CAAACAAGGCTTTTCTCCAAGGG-3′ (SEQ ID NO:6) |
| U6-luc siRNA | 5′-GGATCCAAGGTCGGGCAGGAAGAGGG-3′ (SEQ ID NO:7) |
| (forward) | |
| U6-luc siRNA | 5′-GGATCCAAAAAAGTGCGCTGCTGGT |
| | GCCAACCCTCTCTTGAAGGGTTGGCA |
| | CCAGCAGCGCACGGTGTTTCGTCCTTTCCAC-3′ (SEQ ID NO:8) |

B. Results

The results of the transient transfection of DLD-1 cells with siRNA targeting Kras^{D13}, and the transient transfection of Hela cells with siRNA targeting PLK, as assayed in accordance with the invention, are shown in FIG. 2A. As can be seen, both siRNAs were effective in silencing their gene targets resulting in a substantial reduction in cell growth. The identification of these genes as oncogenes was therefore confirmed.

The results of the stable transduction of DLD-1 cells with the lentiviral vector expressing siRNA targeting Kras^{D13}, as assayed both in a traditional 10-cm dish and in wells of a 96-well plate in accordance with the invention, are shown in FIG. 2B. As can be seen, the assay in accordance with the invention provided results similar to those obtained using the traditional 10-cm plate, and was effective, therefore, in validating the role of Kras^{D13} as an oncogene.

### Example 3

This example shows that the measurement of luciferase activity in cells stably expressing the luciferase gene correlates well with alamarBlue™ readings both in liquid culture and in soft agar.

Two identical series of PC3/luc cells (PC3 cells stably expressing luciferase) at different cell densities were examined in liquid culture. One series was quantified by alamarBlue™ staining and the other by luciferase activity. Luciferase activity was measured in situ by direct and simultaneous addition of cell lysis reagents and luciferase substrates to the culture wells. The results are shown in FIG. 3A (luciferase) and FIG. 3B (alamarBlue™). These demonstrate that luciferase activity in liquid culture exhibits a nearly linear relationship to the number of cells plated, similar to that measurable using alamarBlue™ staining.

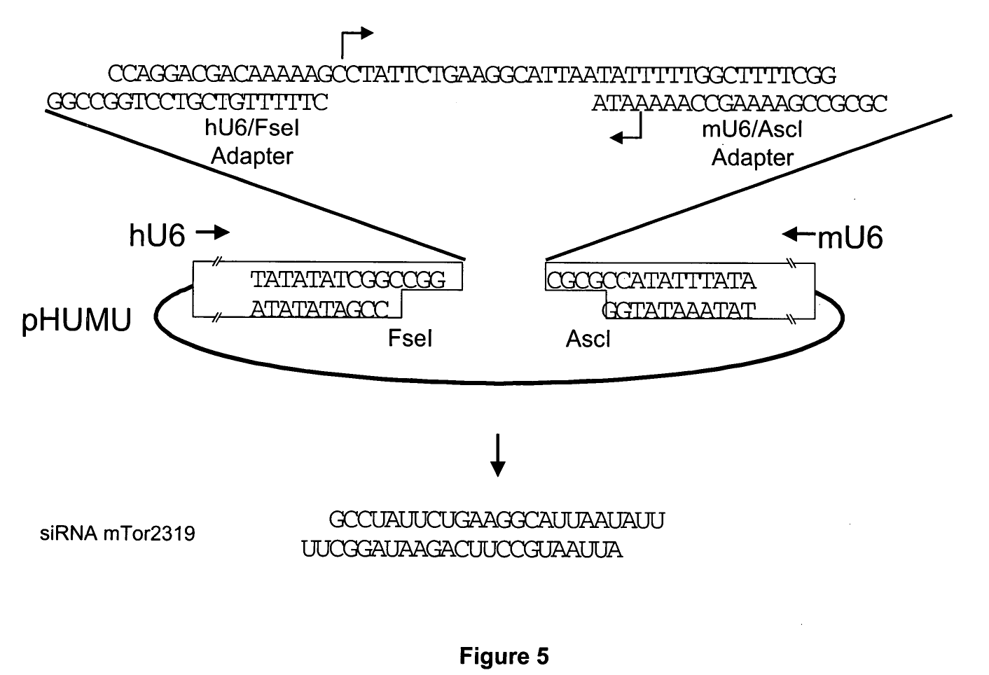

To determine whether measurement of in situ luciferase activity may also be effective in the 96-well soft agar growth assay, PC3/luc cells were plated in 96 well soft agar at two identical serial cell densities (in a manner similar to that described above for liquid culture). AlamarBlue™ staining was used as control. The results (FIG. 5) indicate that the lysis of cells in soft agar was efficient and that luciferase activity was linearly correlated with cell number. Furthermore, the luciferase reading had a significantly lower background as compared to the alamarBlue™ readings (FIGS. 3B and 4B), and thus the detection of live cells by luciferase readings is even more sensitive than that of alamarBlue.

### Example 4

This example shows that cancer cell growth/survival may be efficiently measured in accordance with the invention by co-transfecting cancer cells with a vector expressing siRNA and a reporter gene expressing luciferase or β-galactosidase (LacZ).

A. Materials and Methods

Cells and Vectors

HeLa and A549 cell lines were cultured in Dulbecco's Modified Eagle's Medium (DMEM, Invitrogen, Carlsbad, Calif.), supplemented with 10% Fetal Bovine Serum (FBS, Invitrogen),. 2 mM L-Glutamine (L-Glu, Fisher Scientific), 1×Non-Essential Amino Acids (NEAA, Irvine Scientific, Santa Ana, Calif.), and 1% Sodium Pyruvate, (Invitrogen). PC3/luc cells (PC3 cells containing full-length luciferase cDNA) were maintained in RPMI 1640 (Invitrogen) supplemented with 10% FBS and 2 mM L-Glu. Cells were maintained in a humidified incubator with 5% CO₂ at 37° C.

The luciferase reporter gene expression vector (pGL3-control) and the β-galactosidase (lacZ) reporter gene expression vector (CMV-lacZ) were purchased from Promega (Madison, Wis.). The pcDNA3-Bax expression construct was obtained from ScienceReagents, Inc. (El Cajon, Calif.).

Vector Construction

An siRNA expression vector comprising opposing human and murine U6 promoters and unique FseI/AscI restriction sites for insertion of the siRNA coding sequence was constructed as follows. A modified human U6 promoter was amplified from pTZ U6+1 (Lee, et al., Nat. Biotechnol., 20:500-505 (2002)) by PCR using oligonucleotides 5′-TGCTGGATCCAAGCTTAAGGTCGGGCAGGAAGAG-3′ (SEQ ID NO:9) and 5′-GCATGCTCGAGGCCGGCCGATATATAAAGCCAAGAAATCG-3′ (SEQ ID NO:10) as primers. A modified murine U6 promoter was amplified from pSilencer (Ambion, Austin, Tex.) by PCR using oligonucleotides 5′-TCTAGAGAACTAGTGGATCCGACGCC-3′ (SEQ ID NO:11) and 5′ -GCCGCTCGAGGCGCGCCATATTTATAGTCTCAAAACACAC-3′ (SEQ ID NO:12) as primers. The PCR products were ligated into pCR-Blunt II-TOPO (Invitrogen, Carlsbad, Calif.). The 315 bp XhoI/XbaI fragment from one of the resulting murine U6 promoter vectors was ligated into the 3.8 kb XhoI/XbaI fragment from one of the resulting human U6 promoter vectors in which the two BamHI sites were 47 bp apart. The human U6/murine U6 opposing promoter cassette was isolated from the resulting vector as a 563 bp BamHI/BamHI fragment and ligated into BamHI digested pBluescript II KS to yield pHUMU (FIG. 5).

Opposing promoter vectors for expression of siRNAs against mTOR, Hsp90, and PLK were constructed as follows. Oligonucleotides comprising the sense siRNA strand sequence flanked at the 5′ end by the sequence 5′-pCCAGGACGACAAAAA-3′ (SEQ ID NO:13) and at the 3′ end by the sequence 5′-ATTTTTGGCTTTTCGG-3′ (SEQ ID NO:14) were synthesized by IDT (Coralville, Iowa). These oligos were annealed to adaptor oligos hU6/FseI (5′-CTTTTTGTCGTCCTGGCCGG-3′; (SEQ ID NO:15)) and mU6/AscI (5′-pCGCGCCGAAAAGCCAAAAATA-3′; (SEQ ID NO:16)) and ligated into FseI/AscI-digested PHUMU (FIG. 5). The ligated vectors were transformed into bacteria where the single-stranded region encoding the antisense siRNA strand was filled in by bacterial polymerases. The following siRNA sense strand sequences were used to generate these vectors: mTOR244, GAGAGGAAAGGTGGCATCT (SEQ ID NO:17); mTOR3072, GAGCCACATCAGACCTTATAT (SEQ ID NO:18); mTOR2319, GCCTATTCTGAAGGCATTAAT (SEQ ID NO:19); Hsp90β2035, gCGCATCTATCGCATGATCAAt (SEQ ID NO:20); Hsp90β782, GCGGTAAGGATAAGAAGAAt (SEQ ID NO:21); Hsp90β192, GTTGGACAGTGGTAAAGAGCT (SEQ ID NO:22); PLK772, GAGACCTACCTCCGGATCAt (SEQ ID NO:23); PLK1345, GACAGCCTGCAGTACATAGAt (SEQ ID NO:24); and PLK713, GGTGTATCATGTATACCTTGT (SEQ ID NO:25). When necessary, a non-homologous residue (indicated in lower case) was inserted at the 5′ or 3′ end of the siRNA sense strand sequence due to the preference of U6 promoters for purine residues at the transcription start site.

Co-transfection of luciferase or lacZ Reporter Gene with siRNA and cDNA Expression Vectors

Luciferase or lacZ gene expression cassette vectors were co-transfected with expression vectors for siRNAs targeting mTOR, Hsp90β, PLK, or luciferase; a non-targeting control siRNA, or Bax cDNA using TransIT-LT1 transfection reagent (Mirus, Madison, Wis.) according to the manufacturer's instructions. Briefly, for HeLa cells, 0.05 μg pGL3-control, 0.05 μg of CMV-lacZ and 0.1 μg siRNA or cDNA expression vectors were mixed with 0.6 _l/well of TransIT-LT1 in 15 μl of Opti-MEM (Invitrogen) in 96 well plates and transfected into 3.0×10³ freshly detached cell suspensions. For A549 cell, 0.1 μg pGL3-control, and 0.1 μg siRNA or cDNA expression vectors were transfected into 3×10³cells/well. Three days post-transfection, cell growth rate was measured.

B. Results

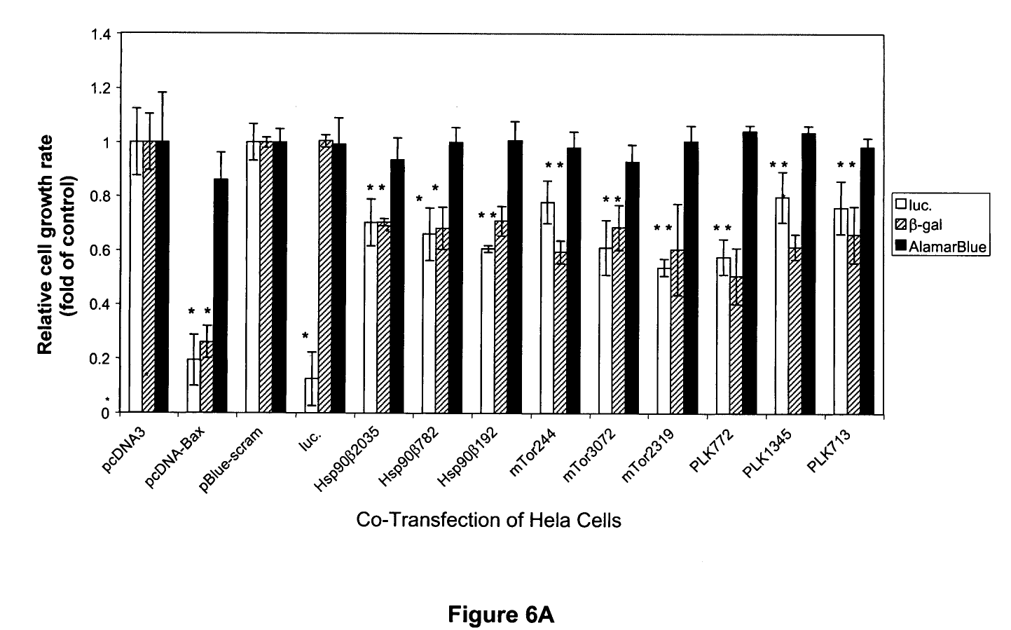

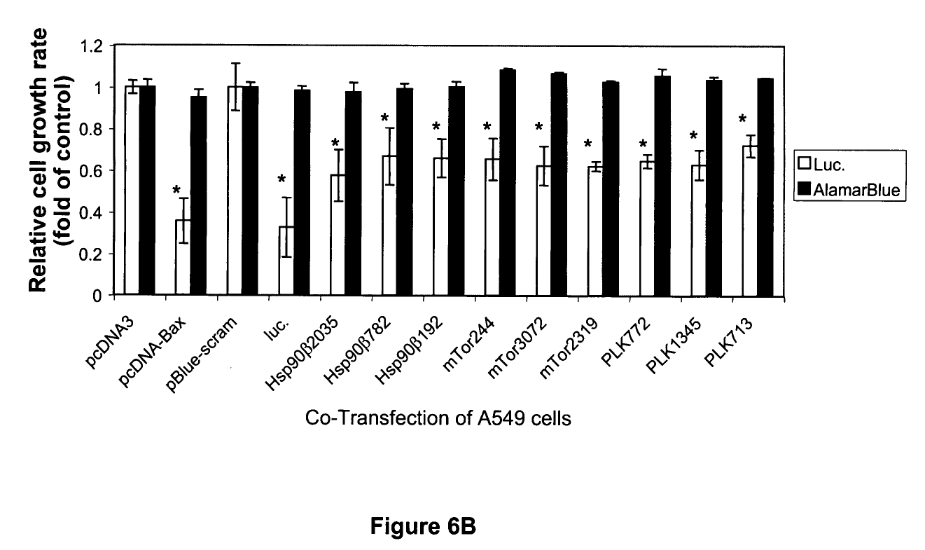

The results of the co-transfection of Hela cells are shown in FIG. 6A and the results of the co-transfection of A549 cells are shown in FIG. 6B.

As can be seen from both figures, when the cells were co-transfected with siRNA against luciferase and with the reporter gene expressing luciferase (both figures) or with β-gal (FIG. 6A only), a 90% reduction of luciferase activity was measured ((as compared to the cells expressing non-specific siRNA (p<0.01)). This result indicates that the majority of the transfected cells acquired both the siRNA vector and the reporter gene vector. In addition, since there was no change in β-galactosidase activity, the gene silencing was luciferase-specific, and did not result from any other factors associated with the transfection process.

PLK has been shown to play an important role in cell cycle progression, and its elevated expression is correlated with the development of many different types of cancers. Depletion of PLK by siRNA in cancer cells results in inhibited cell proliferation, decreased viability and cell-cycle arrest. Liu and Erikson, Proc Natl Acad Sci USA, 100:5789-94 (2003). mTOR is a serine/threonine kinase that functions downstream from Akt to regulate cell growth and proliferation. Rapamycin, a specific inhibitor of mTOR, potently inhibits cell proliferation in a number of cancer types, suggesting mTOR is a potential anti-cancer target. Bjornsti and Houghton, Nat Rev Cancer, 4:335-48 (2004). Hsp90 is a molecular chaperone that regulates stability of a number of client proteins including several oncogenic proteins. Inhibition of Hsp90 by small molecular inhibitors suppresses cancer cell growth and delays cancer cell cycle progress. Neckers and Ivy, Curr Opin Oncol., 15:419-24 (2003). Bax is a well-known tumor suppressor, belonging to the Bcl-2 family of proapoptotic genes. Ranger et al., Nat Genet., 28:113-8 (2001). It has been shown to cause massive cell apoptosis when over-expressed in many cell lines.

When either Hela cells (FIG. 6A) or A549 cells (FIG. 6B) were transiently transfected with siRNAs against these three oncogene targets, or with a vector expressing Bax, measurement by alamarBlue™ staining showed little, if any, effect on cell proliferation, presumably due to the low transfection efficiency. In contrast, when these cells were co-transfected either with luciferase or β-galactosidase, and their activity measured in accordance with the invention, a significant and consistent reduction in proliferation was seen (20-40% reduction, p<0.05). These results are consistent with the oncogene and tumor suppressor properties of these targets, and demonstrate that reduced cell proliferation by siRNA vectors is easily detected using the luciferase or β-galactosidase reporter assays.

FIG. 6B also shows that similar results may be obtained when using the assay of the present invention in connection with cell lines that are less transfectable than Hela. A549 is a non-small cell lung carcinoma cell line and is known to be much less transfectable as compared to HeLa. Nevertheless, as can be seen from FIG. 6B, reduction in luciferase activity was readily detected (20% to 60%, p<0.05) similar to that observed in HeLa cells (FIG. 6A). Similar observations were also made regarding HOP62 and NIH-H460, two non-small cell lung cancer cell lines, which are also less transfectable than HeLa cells. These results are consistent with the prediction that the relative live cell numbers reflected by surrogate activities such as luciferase or β-gal, are independent of transfection efficiency. Furthermore, the fact that severe reduction in luciferase activity was detected in the Bax co-transfection experiment indicates that cells undergoing apoptosis contribute little to the luciferase readings.

All references cited herein are fully incorporated by reference.

We claim:

1. An assay for identifying and/or validating a gene involved in cancer, the assay comprising: (i) a substrate having a multiplicity of wells, each well having a semi-solid medium therein; (ii) cancer cells seeded in the semi-solid medium; (iii) an agent that interacts with the cancer cells; (iv) a detectable marker; and (v) means for automatically quantifying cell survival resulting from the interaction between the agent and the cancer cells.

2. The assay of claim 1, wherein the agent is a nucleic acid molecule selected from the group consisting of a ribozyme, an antisense molecule and an siRNA.

3. The assay of claim 1, wherein the detectable marker is selected from the group consisting of alamarBlue™, tertazolium salt and WST-1.

4. The assay of claim 1, wherein the agent is siRNA and the detectable marker is expressed by a reporter gene co-transfected into the cells together with the siRNA.

5. The assay of claim 4, wherein the reporter gene expresses luciferase or β-galactosidase.

6. The assay of claim 1, wherein the means for automatically quantifying cell survival is fluorometric or calorimetric.

7. The assay of claim 1, wherein the substrate is a microplate comprising 96 wells.

8. The assay of claim 1, wherein the substrate is a microplate comprising between 6 to 384 wells.

9. An assay for identifying and/or validating a compound as a therapeutic agent for the treatment of cancer, the assay comprising: (i) a substrate having a multiplicity of wells, each well having a semi-solid medium therein; (ii) cancer cells seeded in the semi-solid medium; (iii) a compound that interacts with the cancer cells; (iv) a detectable marker; and (v) means for automatically quantifying cell survival resulting from the interaction between the compound and the cancer cells.

10. The assay of claim 9, wherein the detectable marker is selected from the group consisting of alamarBlue™, tertazolium salt and WST-1.

11. The assay of claim 9, wherein the means for automatically quantifying cell survival is fluorometric or calorimetric.

12. The assay of claim 9, wherein the substrate is a microplate comprising 96 wells.

13. The assay of claim 9, wherein the substrate is a microplate comprising between 6 to 384 wells.

14. A high throughput screening method for identifying and/or validating a gene involved in cancer growth, the method comprising: (a) seeding cancer cells in a semi-solid medium, within the wells of a substrate having a multiplicity of wells; (b) introducing into the wells a detectable marker and an agent that interacts with the cancer cells; (c) allowing the cells to grow for between about 5 to about 15 days; and (d) obtaining automatically a quantifiable measure of cell survival resulting from the interaction between the agent and the cancer cells.

15. The method of claim 14, wherein the agent is a nucleic acid molecule selected from the group consisting of a ribozyme, an antisense molecule and an siRNA.

16. The method of claim 14, wherein the detectable marker is selected from the group consisting of alamarBlue™, tertazolium salt and WST-1.

17. The method of claim 14, wherein the agent is siRNA and the detectable marker is expressed by a reporter gene co-transfected into the cells together with the siRNA.

18. The method of claim 17, wherein the reporter gene expresses luciferase or β-galactosidase.

19. The method of claim 14, wherein the step of automatically obtaining a quantifiable measure of cell survival is fluorometric or calorimetric.

20. The method of claim 14, wherein the substrate is a microplate comprising 96 wells.

21. The method of claim 14, wherein the substrate is a microplate comprising between 6 to 384 wells.

22. The method of claim 14, wherein the cells are grown from about 7 to about 15 days.

23. A high throughput screening method for identifying and/or validating a compound as therapeutic agent for the treatment of cancer, the method comprising: (a) seeding cancer cells in a semi-solid medium, within the wells of a substrate having a multiplicity of wells; (b) introducing into the wells a detectable marker and a compound that interacts with the cancer cells; (c) allowing the cells to grow for between about 5 to about 15 days; and (d) obtaining automatically a quantifiable measure of cell survival or cell growth resulting from the interaction between the compound and the cancer cells.

24. The method of claim 23, wherein the detectable marker is selected from the group consisting of alamarBlue™, tertazolium salt and WST-1.

25. The method of claim 23, wherein the step of automatically obtaining a quantifiable measure of cell survival is fluorometric or calorimetric.

26. The method of claim 23, wherein the substrate is a microplate comprising 96 wells.

27. The method of claim 23, wherein the substrate is a microplate comprising between 6 to 384 wells.

28. The method of claim 23, wherein the cells are grown from about 7 to about 15 days.
